(19) United States
(12) Patent Application Publication
(10) Pub. No.: US 2016/0000878 A1
(43) Pub. Date: Jan. 7, 2016

(54) Treatment of Prior Disease With Oral Interferon Alpha

(71) Applicant: Joseph Cummins, Edmond, OK (US)

(72) Inventor: Joseph Cummins, Edmond, OK (US)

(21) Appl. No.: 14/791,889

(22) Filed: Jul. 6, 2015

Related U.S. Application Data
(60) Provisional application No. 62/021,004, filed on Jul. 3, 2014.

(51) Int. Cl. A61K 38/21; A23K 1/16; A23K 1/18; A61K 47/26

(52) U.S. Cl. CPC A61K 38/212; A61K 47/26; A23K 1/1643; A23K 1/1631; A23K 1/1813

(57) ABSTRACT

The present disclosure is directed to compositions and methods for treating prion diseases through oral administration of low doses of interferon-alpha. In one embodiment a composition is administered comprising interferon-alpha and trehalose.

### CROSS-REFERENCE TO RELATED APPLICATIONS

This application claims the benefit of U.S. patent application Ser. No. 62/021,004, a provisional utility application filed on 07/03/14. This application is made on the first USPTO business day following the anniversary of that earlier application and is therefore timely made.

### STATEMENT REGARDING FEDERALLY SPONSORED RESEARCH OR DEVELOPMENT (IF APPLICABLE)

Not applicable.

### REFERENCE TO SEQUENCE LISTING, A TABLE, OR A COMPUTER PROGRAM LISTING COMPACT DISC APPENDIX (IF APPLICABLE)

Not applicable.

### BACKGROUND OF THE INVENTION

This disclosure relates generally to a treatment of prior disease with oral interferon alpha. None of the known inventions and patents, taken either singularly or in combination, is seen to describe the instant disclosure as claim ed. Accordingly, an improved treatment of prior disease with oral interferon alpha would be advantageous.

### BRIEF SUMMARY OF THE INVENTION

### BRIEF DESCRIPTION OF THE SEVERAL VIEWS OF THE DRAWING

Not applicable.

### DETAILED DESCRIPTION OF THE INVENTION

Described herein is a treatment of prion disease with oral interferon alpha. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claim s appended hereto are not intended to be limited by the disclosed embodiments, but are to be accorded their widest scope consistent with the principles and features disclosed herein.

Protein conformational diseases include a variety of unrelated diseases that arise from aberrant conformational transition of a protein, which in turn leads to self-association of the aberrant protein forms, with consequent tissue deposition and damage. These diseases also share striking similarities in clinical presentations, typically a rapid progression from diagnosis to death following varying lengths of incubation.

One group of conformational diseases is termed “prion diseases” or “transmissible spongiform encephalopathies (TSEs).” In humans, these diseases include Creutzfeldt-Jakob disease (CJD), Gerstmann-Straussler-Scheinker syndrome (GSS), Fatal Familial Insomnia, and Kuru. In animals the TSE's include scrapie of sheep, bovine spongiform encephalopathy (BSE), transmissible mink encephalopathy (TME), and chronic wasting disease (CWD) of captive and free-ranging mule deer, whitetail deer and elk. All of the TSEs are characterized by the accumulation of abnormal (beta pleated sheet-rich, proteinase K resistant) permanent conformational change in a normal cell (c) protein, termed the prion protein (PrP or cPrP). Virtually all workers in the TSE field subscribe to the “prion hypothesis”. In this view, the TSEs are due to a self-replicating or self propagating mutant PrP within the neuropil of affected individuals. This aberrant protein, PrP scrapie (PrPsc) is certainly a central feature of the TSEs, but the data regarding infectivity and self-replication as the necessary and central infectious principle of the TSEs is not definitive. In experimental animal studies, the PrP accumulates first in lymphoid tissues and then the neuropil. Progressive accumulation of PrP within neurons results in intracellular vacuolation and edema (spongiform changes) and neuronal necrosis with reactive astrocytic gliosis ultimately results in fatal central nervous system disease. While this sequence of events is not in dispute, the actual infectivity of PrPs for the CNS has not been formally proven. In the TSEs, evidence is accumulating for the presence of a structurally distinct virion-like particle (VLP) as the fundamental infectious etiology of the TSEs (Manuelidis, Laura. A 25 nm Virion is the likely cause of Transmissible Spongiform Encephalopathies. J. Cell. Biochem. 100:897-915, 2007). Specifically, TSE infectivity and transmissibility is separable from PrP production. TSE infectivity is inhibited by in vitro virus interference assays and the TSE agent can be propagated in vitro, in the absence of PrP production. VLP-TSE infectivity is susceptible to nuclease digestion and to denaturation by strong detergents whereas the PrPs are not. Ultrastructurally, VLPs are regularly identified in TSE tissues as VLP particles of 25-35 nm in diameter, roughly the size of ribosomes. The VLPs can be propagated in vitro and are infectious and pathogenic (induce TSE disease) in experimental laboratory animal species. (Manuelidis, Laura. A 25 nm Virion is the likely cause of Transmissible Spongiform Encephalopathies. J. Cell. Biochem. 100:897-915, 2007). While the identity of the TSE-VLPs is still not defined, physical characteristics (nonenveloped and resistant to pH extremes), ultrastructural features (small icosohedral virions), cytoplasmic location and size (genome estimated at <5,000 bases) strongly argue that the VLPs are members of, or similar to the torque teno viruses (TTVs), a recently discovered group human and animal viruses.

The PrPres is derived from a constitutive normal cell called the prion protein (PrPc); optical spectroscopy and crystallography studies have revealed that the TSE-related form(s) of prions are sub fibrillar proteins with a beta-pleated sheet structure virtually indistinguishable from amyloid fibril deposits. In contrast, the normal homologue to this protein, the PrPc has an alpha-helical folded 3-dimensional tertiary structure. (See, e.g., Wille et al. (2001) Proc. Nat'l Acad. Sci. USA 99:3563-3568; Peretz et al. (1997) J. Mol. Biol. 273:614-622; Cohen & Prusiner, Chapter 5: Structural Studies of Prion Proteins in PRION BIOLOGY AND DISEASES, ed. S. Prusiner, Cold Spring Harbor Laboratory Press, 1999, pp: 191-228). The resultant three-dimensional change in the putative PrPse alters the biochemical properties of normal PrPc. Specifically, the PrPc is soluble in non-denaturing detergents whereas the TSEPrPres are insoluble in these same detergents. The PrPc is readily digested by proteases, while TSE-PrPres is partially resistant to proteolytic digestion. Proteolytic enzyme resistance results in the generation of partially degraded TSE-PrPsc fragment(s) identified as PrP 27-30 kDa or the proteinase K (PK) PK-resistant TSE-PrPes. Finally, abnormal TSE-PrPres may convert normal cellular PrPc to the pathogenic beta-pleated sheet conformational form. (See, e.g., Kaneko et al. (1995) Proc. Nat'l Acad. Sci. USA 92:11160-11164; Caughey (2003)) Br Med Bull. 66:109-20. The presence of the disease-related form of PrPc (i.e. PrPres) in tissues, is a widely accepted specific molecular marker of the TSE disease and inhibition of PrPres accumulation is frequently used to evaluate the efficacy of therapeutic drugs proposed for therapy of the TSEs.

Two general approaches to the therapy of the TSEs are available. The first of these involve drug-induced alternations (levels, conformational changes, etc.) in the hallmark protein of the TSEs, PrPsc. The second general approach addresses the etiologic cause of the TSEs, the VLPs. Progress in the former has been made and these are described below. Progress in the latter is in it's infancy but there are important clues to a successful approach to for anti-viral therapy directed against the putative VLP of the TSEs.

Drug Therapies for the TSE-induced diseases:

To date, several compounds have been described which decrease the PrPres concentration in different scrapie-infected cell lines or prolong the incubation period in animal models. These drugs belong to different classes, including sulfated polyanions, amphotericin B derivatives, Congo red, tetracyclic compounds, tetrapyrroles, branched polyamines, b-sheet breakers derived from PrP peptides, quinacrine, chlorpromazine, and some tricyclic derivatives with an aliphatic side chain. However, these compounds are maximally effective when given at clinical onset of TSE and the limited efficacy of these compounds do not support an evidence-based rationale for their use in the treatment of the TSEs.

Two US patents have issued (U.S. Pat. Nos. 6,270,954 and 5,900,360) disclosing the use of protein stabilizing agents, including glycine, alanine, proline, taurine, betaine, octopine, glutamate, sarcosine, gamma-aminobutyric acid, glycerol, trimethylamine N-oxide (TMAO), erythritol, trehalose, isofluoroside, and polyethylene glycol to improve the conformational defect in cells with defective PrPc. In addition, the oral administration of disaccharides, such as trehalose, has been reported to decrease polyglutamine aggregates in cerebrum and liver, improve motor function and extend lifespan in a transgenic mouse model of Huntington disease. These beneficial effects are believed to be the result of trehalose binding to expanded polyglutamines and stabilizing the partially unfolded polyglutamine-containing protein. Low toxicity profiles and high solubility, coupled with efficacy upon oral administration, make trehalose promising as a therapeutic drug or lead compound for the treatment of polyglutamine diseases.

The ubiquitin-proteasome system is the primary mechanism utilized in eukaryotic cells for degrading unwanted and miss-folded proteins that have been “tagged” with ubiquitin, a highly conserved 76-amino acid polypeptide. The ubiquitin-proteasome system is a multiprotein complex that can specifically degrade polyubiquinated proteins into short polypeptides and amino acids in an ATP-driven reaction. The 26S proteasome is composed of two subcomplexes: A 20S core catalytic moiety and a regulatory 19S moiety. Degradation of a protein via the ubiquitin-proteasome pathway (UPP) is accomplished by two discrete and successive steps: 1) Tagging of the substrate by covalent attachment of ubiquitin molecules to synthesize the poly-ubiquitin chain proteolytic signal, and, 2) Degradation of the tagged protein by the 26S proteasome complex release of free peptides and ubiquitin catalyzed by ubiquitin-recycling enzymes.

More recently, a sizeable group of ubiquitin-related proteins have been described including at least a dozen distinct ubiquitin-like (UbL) proteins. Similar to ubiquitin, these ubiquitin-related proteins form covalent attachments to other macromolecules. The Ubls mediate an impressive range of cellular functions, including cell-cycle progression, DNA repair, and apoptosis, suggesting that covalent post-translational modification of proteins is a versatile principle of determining the half-life, intracellular localization, and activity of proteins. An important member of the Ubl is the protein product of IFN-stimulated gene 15 (ISG-15). After interferon exposure, ISG-15 is one of the most strongly induced cellular interferon response genes. ISG-15 is also induced by influenza B virus, lipopolysaccharide, and the cellular stress response. The ISG-15 protein product contains two ubiquitin homology domains and conjugates a variety of proteins when cells are treated with type 1 interferon or lipopolysaccharide. Although ISG-15 protein shares several properties in common with other Ubls, it is unique in that expression and conjugation to target proteins is tightly regulated by specific signaling pathways, that are strongly associated with various functions of the innate immune system. Important to this patent application is the fact that low dose orally-administered interferon upregulates ISG-15 gene expression (see J. K. Smith et al Journal of Interferon and Cytokine Research 19:923-928, 1999) and also the expression of the 26S proteasome (see Example 1).

Anti-TSE-VLP Therapy

While the identity of the TSE-VLP is unresolved, specific characteristics of the TSE30 VLP, compatible with over a decade of findings on the nature of the VLPs, are listed below. The TSE-VLP is a small nonenveloped particle of 30-35 nm diameter that can encapsidate a genome of roughly 5,000 bases (or base pairs). The putative genome has the potential to encode one structural (nucleocapsid) protein and one viral DNA replicase enzyme. The agent is infectious by conventional means, replicates in and expands within lymphoreticular tissues, specifically macrophage-lineage cells during the subclinical phase of infection and has a species-specific or at least limited host range. While genomically stable, it retains the capacity for mutation(s) and needs host cell PrP for some aspect of its replicative cycle. Within microglia and neuropil, the host-TSE-VLP interaction is expressed predominately as a noncytolytic infection cycle with cellular degenerative changes (cytoplasmic vacuoles and PrP-amyloid deposits) that are not immediately cytotoxic to infected cells. Since these degenerative cellular changes are progressive over time, the end result is progressive neuron loss, astrocytic and microglial gliosis and slowly developing atrophy of affected regions (chiefly the cerebral cortex) of the CNS.

Of all of the viral agents known to infect humans and animals, the identity of the putative TSE-VLP is most compatible with the known characteristics of the torque teno viruses (TTVs) or a closely related and as yet unidentified viral genus. Since discovery in 1997, the TTVs/TTMVs have been well studied at the molecular level; the complete nucleotide sequence of many TTVs (human, primate & porcine) is known. Definitive studies are hampered by the fact that satisfactory conventional methods for in vitro propagation of the TTVs are not available; direct and nested (n) PCR are the only reliable tools for detection of TTV DNAs in patient samples. Human (h) TTVs are circular single-stranded (ss) DNA molecules of 3,500-3,800 bases; the hTTMVs are smaller (2,800 bases) and, aside from a common GC-rich initiation sequence, hTTVs and hTTMVs share very little sequence identity. The nonenveloped virion icosohedral particle size is 30-35 nm in diameter and the TTVs have been provisionally assigned to the Anellovirus genus within the Circoviridae family.

The Anelloviruses, like the other Circoviridae, employ “rolling circle” amplification to replicate DNA. Viral complementary DNA is generated by the host nuclear DNA polymerases during the cell cycle S phase to create double-stranded (ds) DNA replicative intermediate isoform(s). TTV-DNA replication is initiated through homologous recombination at the “stem loop”-equivalent site in the untranslated region or UTR. Like CAV, the TTVs lack the conserved “stem loop” transcription start site (nucleotides 342-349) characteristic of other Circoviridae; TTV-DNA transcription proceeds from a GC-rich area in the UTR. Unlike the other Circoviridae whose ORF coding orientation proceeds in the ambi-sense direction, the antisense TTV strand is used for TTV transcription and is also encapsidated. TTV DNA contains three overlapping ORFs and transcribes six (three spliced) mRNAs that code for proteins of predicted 770 (ORF1), 202 (ORF2) and 105 (ORF3) amino acids (AA) respectively. The ORF1 gene product, viral protein 1 (VP1) is the viral nucleocapsid protein. VP1 is strongly associated with virion DNA by an N-terminal DNA binding motif and is the only structural protein of the virion. VP1 (ORF1) is the most “significant” viral protein since this protein that contains hypervariable regions and interacts with TTV-DNA, host cells and antibodies. The function(s) of TTV VP2 and VP3 are not yet formally assigned. By analogy to CAV (36,37), VP2 is a nonstructural chaperonin protein with protein phosphatase activity; VP3 has apoptonin-like activity and likely is involved in initiation of viral replication. The TTMV genome is similarly organized but smaller. Roughly 25% of the TTVMV genome is UTR; the TTMV VP1 is 675 AA; VP2 is 100 AA. In spite of the detailed knowledge of the molecular biology of the Anellovirus group achieved principally via molecular biology techniques, the TTVs are unculturable in vitro and a satisfactory in vitro culture system for the TTVs does not exist.

Within TTV-infected subjects, viral DNAs, replicative isoform DNAs and viral nucleocapsid protein has been demonstrated in phagocytic mononuclear cells of bone marrow, peripheral blood and in tissues. Like the scrapie agent and other TSE-VLPs, replication of TTVs is prominent in bone morrow and spleen. Sequence analyses have revealed remarkable genomic diversity amongst the human (h) TTVs. Individual isolates may vary by greater than 40% of their genomic sequence. Diversity is expressed between isolates recovered from different people and within infected individuals over time, indicating that the hTTVs behave as “quasi-species” both within and between infected people (Nishizawa et al, J Virol, 73:9604-9608, 1999). The greatest sequence diversity is located in two hyper-variable regions in the ORF1 gene and the UTR. The hTTVs consist of at least six genogroups; within each genogroup, numerous genotypes have been identified. Genogroup A TTV contains the prototype hTTV and several other human TTVs. Group B hTTVs are found in humans and primates. Group C hTTVs are found in humans only and Group D TTVs are found in chimpanzees only. Information on the distribution of Groups E and F hTTVs are not available. There are three genogroups in the TTMV family. Porcine TTVs are divided into two genogroups and are also genetically heterogeneous, particularly in the UTR. Little is known about ruminant TTVs.

The hTTVs and hTTMVs DNAs have been found in sera/plasma and tissues of many subjects. The TTVs are widely distributed in human and animal populations; it is not uncommon for a single patient to be actively infected with multiple hTTV genogroups. The prevalence of TTV viremia rises in early childhood and increases with age to adulthood. Human TTVs have zoonotic potential since they readily infect primates. Captive primates are TTV-positive and contain TTVs of both human and primate origin. Free-living wild primates (no human contacts) are also primate- TTV-positive. In swine, surveys indicate that there is also a high incidence (40-100%) of sTTV viremia. In utero transmission has been demonstrated in humans and in swine. Patients receiving human blood and blood products, intravenous drug abusers, etc., are recognized as high-risk categories for acquiring hTTVs. Although the dominant mode of TTV transmission has not been formally defined, the fecal-oral route is an important natural means of infection. Infective aerosols are implicated transmission; hTTVs have been demonstrated in lungs, nasal secretions and saliva.

Most workers in the TTV field believe that the TTVs are-species specific viral agents. However, this is not completely true as hTTV is readily transmitted (contact or by injection) to primates (captive and free-living) as an asymptomatic infection. The TSE-VLP may be an, as yet undescribed and novel genogroup(s) of hTTVs. Alternatively, the TSE-VLP in humans may be TTV species of domestic animals (swine, sheep or cattle) that have adapted to replication in human hosts via the process of genomic mutation(s). The clearest evidence for a direct connection between ruminants and humans is the BSE outbreak in cattle and in humans who consumed BSE-positive tissues in the early 1990's in the United Kingdom. Presumably, the BSE agent, originally confined to sheep as scrapie, was inadvertently adapted to bovids through the widespread practice of feeding inadequately processed scrapie-infected tissues in the form of high protein bone and protein meal to dairy cattle. A similar process resulted in the development of transmissible mink encephalopathy of captive mink in the 1960's in the USA. Thus, while definitive linkage of TSE-VLP to TTVs or TTV-like viruses has not been accomplished, supportive and circumstantial data is persuasive.

Several reports highlight the susceptibility of hTTV to interferon alpha therapy (see for example, Maggi et al. J Virol, 75(24):11999-2004, 2001; Lai et al, World J Gastroenterol, 8(3):567-70, 2002; and Dai et al, Antiviral Res, 53(1):9-18, 2002). Investigators noted that patients being treated for inflammatory liver disease with IFNa became hTTV-DNA-negative following therapy. As well, we have previously shown that low dose oral interferon therapy is effective against several viral infectious diseases of swine including rotavirus (RNA virus), transmissible gastroenteritis virus (DNA virus), porcine reproductive and respiratory syndrome virus (RNA virus) and porcine circovirus type 2 (DNA virus). The last is in the same viral family (Circoviridiae, genus Circovirus) as are the TTVs (Circoviridiae, genus Annellovirus).

Originally identified for their ability to induce cellular resistance to viral infection, IFNs are currently known to be potent mediators in the host defense mechanism and homeostasis, modulating both the innate and adaptive immune responses. IFNs are small, inducible 20-25 K, usually glycosylated proteins that are produced by vertebrate cells in response to various biological stimuli. Mechanistically, IFNs mediate their biological activities by binding to receptors present on the surface of target cells. Specific ligand-receptor interactions trigger intracellular signaling cascade downstream, resulting in the synthesis of proteins that mediate mentioned pleiotropic activities.

“Interferon” is a term generically describing a distinct group of cytokines exhibiting pleiotropic activity generally categorized as antiviral, antiproliferative and immunomodulatory. In the early years of interferon research, an international committee was assembled to devise a system for orderly nomenclature of the various interferon species and defined “interferon” as follows: To qualify as an interferon, a factor must be a protein which exerts virus non-specific, antiviral activity at least in homologous cells through cellular metabolic process involving synthesis of both RNA and protein. “Interferon” as used herein in describing the present invention shall be deemed to have that definition and shall contemplate such proteins and glycoproteins, including for example, the subtypes IFN-α, IFN-β, IFN-δ, IFN-γ, IFN-ε, IFN-κ, IFN-λ, IFN-ω and IFN-tau, regardless of their source or method of preparation or isolation.

Based structure, physicochemical properties and biological activities, the IFNs are classified into two major groups: type I or type II. In mammals, eight families of type I IFN have been described. These are: IFN-α, IFN-β, IFN-δ, IFN-ε, IFN-κ, IFN-λ, IFN-ω and IFN-tau (IFN-τ). A prominent member of the type I IFNs that is germaine to this patent application is IFNa. Among these families, trophoblast IFN-τ, is found only in ruminants and is not virusinduced. Rather it is produced in the embryonic trophoblasts during early pregnancy and its production and actions are needed for successful completion of pregnancy. IFN-δ (delta), a polypeptide of about 149 amino acids, has been described only in pigs. Like IFN-τ above, this IFN is produced by trophoblasts and is associated with successful reproduction in swine species.

As disclosed herein low dose oral interferon alpha can be used to upregulate expression of 26S proteasome and ubiquitin-like molecules such as ISG15 and ubiquitin-like 1 protein (sentrin). The upregulation of such proteins is anticipated to enhance the removal of misfolded proteins and/or slow or prevent the progression and/or transmissibility of a protein conformational disease. Accordingly, the administration of type I interferons can be used to treat prion diseases such as BSE. The effectiveness of interferon containing compositions to treat and/or prevent prion diseases can be further enhanced by the addition of a protein stabilizing agent such as trehalose.

### SUMMARY

In accordance with one embodiment a method of treating prion diseases is provided, comprising the steps of administering low dose interferon to a subject in need of such treatment. In one embodiment the administered interferon is a type I interferon, and more particularly, in one embodiment the interferon is either interferon alpha or interferon beta and the interferon is administered orally. Administration of low dose interferon will 1) promote enhanced removal of PrPsc as they are formed and 2) will have a direct anti-viral effect on the VLP of the TSEs. Furthermore, the interferon can be combined with additional active agents such as an osmolytic agent. In one embodiment the interferon composition comprises interferon-alpha, anhydrous crystalline maltose and trehalose. For administration to animals, the compositions can be further admixed with standard animal feed formulations. For treating a ruminant species for a prion disease, for example treating cattle for BSE, the interferon-containing composition can be encapsulated or otherwise modified to protect the active agents as the orally administered formulation passes through the rumen.

DEFINITIONS In describing and claiming the invention, the following terminology will be used in accordance with the definitions set forth below.

As used herein, the term “treating” includes prophylaxis of a specific disease or condition, preventing transmission of the disease to others, delaying onset or progression of the disease, or alleviating the symptoms associated with a specific disease or condition and/or preventing or eliminating said symptoms. In the specific case of a prion disease, treating includes inhibiting or preventing the formation of the pathogenic conformation of the protein.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents and includes agents approved by a regulatory agency of the US Federal government or listed in the US Pharmacopeia for use in animals, including humans. The term “carrier” refers to a diluent, adjuvant, excipient or vehicle with which an active agent is administered.

As used herein a “protein stabilizing agent” is a substance that stabilizes a protein in a biologically active conformation and/or inhibits or prevents the formation of conformationally defective protein (e.g. prevents the conversion of PrPc to PrPres). Protein stabilizing agents may also act to induces a conformationally defective target protein to assume a biologically active conformation, or compensates for its inability to assume a biologically active conformation, and causes a conformationally defective target protein to be processed properly, and/or to be targeted properly, and/or to acquire or retain a biological activity similar to that of the wild-type protein.

As used herein, “effective amount” means an amount sufficient to produce a selected effect. For example, an effective amount of a protein stabilizing agent is an amount effective to either stabilizes a protein in a biologically active conformation and/or inhibit or prevent the formation of a conformationally defective protein, resulting in the cells or individual functioning more like the cells or individuals that express the wild type protein.

As used herein, “osmolyte” refers to an agent that lends osmolality to the buffered solution or affects hydration or surface tension. Examples include polyols and sugars such as glycerol, erythritol, arabitol, sorbitol, mannitol, xylitol, mannisidomannitol, glycosyl glycerol, glucose, fructose, sucrose, trehalose, and isofluoroside; polymers such as dextrans, levans, and polyethylene glycol; and some amino acids and derivatives thereof such as glycine, alanine, betaalanine, proline, taurine, betaine, octopine, glutamate, sarcosine, y-aminobutyric acid, and trimethylamine N-oxide (TMAO), as described more fully in Yancey et al., Science, 217:1214-1222 (1982) and Schein, Bio/Technology, 8: 308-315 (1990).

As used herein “upregulation of expression” as it relates to a specific protein is intended to describe an increase cellular concentration of the respective protein/protein complex relative to native or non-stimulated levels. The increased protein levels may arise due to increased expression of the corresponding protein genes or may be due to enhanced stability of the encoded proteins, or a combination thereof

EMBODIMENTS As disclosed herein compositions and methods are provided to treat animals, including humans, suffering from a protein conformational disease, including for example a prion disease. The administration of type 1 interferon can upregulate expression of the 26S proteasome and ubiquitin-like molecules such as ISG15 and sentrin. The upregulation of such proteins is anticipated to enhance the removal of misfolded proteins, and thus slow or prevent the progression and/or transmissibility of a protein conformational disease. Human interferon, given orally in low dosage, upregulated (6.2 times) the expression of proteosome alpha 3 subunit (Namangala et al., J Interferon Cytokine Research 26:675-681, 2006). In accordance with one embodiment a method of treating a patient suffering from a protein conformational disease, including for example a prion disease, is provided. In one embodiment the method comprises the steps of

identifying a patient suffering from a protein conformational disease, including for example a prion disease; and

administering an amount of a composition comprising a low dose type I interferon effective to treat the protein conformational disease, including for example a prion disease.

Accordingly, a method of upregulating the expression of ubiquitin-like molecules, such as ISG15 and/or upregulating the expression of the 26S proteasome in a warm blooded vertebrate is provided as a means of treating a prion disease. In one embodiment the method includes administering a composition comprising an effective amount of type 1 interferon and a pharmaceutically acceptable carrier. In one embodiment the type 1 interferon is interferon-alpha.

The interferon containing composition can be administered to the patient through a number of routes, such as orally, intranasally, inhalation, intramuscularly, or intravenously. In accordance with one embodiment the interferon is administered orally or intranasally. The interferon containing composition can be administered in a single dose, or in several doses per day. In one embodiment, wherein the interferon containing composition is administered orally, the composition is administered in a form or manner that optimizes contact of the composition with the oral and oral pharyngeal mucosa of the animal or human. In one embodiment the interferon containing composition is prepared as a lozenge, powder, liquid or chewable composition. In accordance with one embodiment the interferon is administered in a form of orally dissolving lozenges.

Interferon of human and murine origin is quantified in the art in terms of International Units (IU). Interferons of other than human or murine origin can be used in accordance with this invention. In that presently accepted practices may not extend the use of “International Units” to quantify non-human and non-murine interferons, it shall be understood that administration of amounts of non-human/non-murine interferons having the same efficacy as the quantities (IU's) of human interferon specified in this description are within the scope of the present invention. In one embodiment the interferon is administered orally in a low dosage form.

For the purpose of the present invention, low dosage interferon alpha treatment dosages range from about 0.1 IU/lb to about 100 IU/lb of patient body weight, more typically about 0.5 to about 10 IU/lb of patient body weight. Thus, unit dosage forms for human use typically comprise about 5 IU to about 2500 IU of interferon alpha, more typically about 10 IU to about 300 IU of interferon alpha, in combination with a pharmaceutically acceptable carrier therefor. Dosage forms for treatment in accordance with this invention can be in solid, liquid, aerosol, ointment or cream formulation and are typically administered from one to four times daily until the condition being treated is alleviated. In one embodiment, human alpha interferon is orally administered in a sterile aqueous solution. Chronic administration may be required for sustained benefit. Generally speaking, the dosage forms are administered in a disease state-dependent manner, including particularly administration topically, bucally/sublingually, by oral ingestion or by inhalation.

The interferon containing composition may include additional components such as an osmolyte agent. For example, the interferon containing composition can be further combined with polyols and sugars such as glycerol, erythritol, arabitol, sorbitol, mannitol, xylitol, mannisidomannitol, glycosyl glycerol, glucose, fructose, sucrose, trehalose, and isofluoroside; polymers such as dextrans, levans, and polyethylene glycol; and some amino acids and derivatives thereof such as glycine, alanine, beta-alanine, proline, taurine, betaine, octopine, glutamate, sarcosine, y-aminobutyric acid, and trimethylamine N-oxide (TMAO). In one embodiment the composition comprises interferon-alpha and trehalose. In a further embodiment the interferon containing composition includes a disaccharide selected from the group consisting of maltose, lactose and fructose. In one embodiment the disaccharide is anhydrous crystalline maltose. Anhydrous crystalline maltose can be prepared in accordance with U.S. Pat. No. 4,816,445, the disclosure of which is expressly incorporated herein by reference.

The compositions described herein can be admixed with animal feed for administration to livestock. For example, the present composition can be administered to ungulate species to treat prion diseases. More specifically, interferon containing compositions can be admixed with the animals feed and administered to sheep and cattle, to prevent or minimize the impact of scrapie or BSE on sheep and cattle, respectively. In one embodiment a composition comprising trehalose and oral interferon is blended with standard cattle feed and provided to the animals to minimize BSE infection. Sucram is an intense sweetner and is added to cattle feed because it increases feed intake. Accordingly, it is anticipated that trehalose can substitute this product in animal rations and provide the additional benefits of stabilizing PrPc. In a further embodiment a supplemental animal feed is provided wherein the feed supplement comprises interferon-alpha, a disaccharide selected from the group consisting of maltose, lactose and fructose, and trehalose. This feed supplement is then blended into a standard animal feed formulation. In one embodiment an interferon/anhydrous crystalline maltose (ACM) composition is mixed into trehalose and then that interferon/ACM/trehalose mixture is blended into cattle feed. Alternatively, the interferon composition can be added to the drinking water of the animal or directly administered to the animal.

In one embodiment when the interferon compositions are administered to a ruminant species, the compositions are formulated to bypass the rumen to reach a more favorable digestive environment. Successful bypass of the rumen by an interferon comprising composition can be accomplished using standard microencapsulation technologies known to those skilled in the art. Briefly, the physical methods of encapsulation include spray drying, spray chilling, rotary disk atomization, fluid bed coating, stationary nozzle coextrusion, centrifugal head coextrusion, submerged nozzle coextrusion and pan coating. The chemical methods of encapsulation include phase separation, solvent evaporation, solvent extraction, interfacial polymerization, simple and complex coacervation, in-situ polymerization, liposome technology, and nanoencapsulation. In accordance with one embodiment the active agents (i.e. the interferon composition) are enclosed in an encapsulation system, wherein the encapsulated material has the optimal size and density to move through the rumen of the ruminant before the encapsulating system releases substantial amounts of the active agents. In one embodiment the encapsulated composition provides a controlled, sustained, delayed targeted enteric release of the interferon containing composition.

In accordance with one embodiment a method of stabilizing PrPc protein present in a vertebrate species is provided. The method comprises identifying a vertebrate species in need of PrPc protein stabilization, and administering to such a vertebrate species a composition comprising interferon. In one embodiment the interferon is interferon-alpha that is administered orally in a dosage amount of about 0.1 IU/lb to about 100 IU/lb of animal body weight. The administration of the composition is anticipated to have a prophylactic effect in preventing transmission of the disease and also is anticipated to have use in treating individual animals that have already been infected prior to treatment. In accordance with one embodiment the interferon containing composition further comprises trehalose and a disaccharide selected from the group consisting of maltose, lactose and fructose. In one embodiment the disaccharide is anhydrous crystalline maltose.

### EXAMPLE 1

Interferon was administered to a human patient and blood samples were taken before and after administration of interferon-alpha to determine what blood components were affected by the oral administration of interferon alpha. Table 1 provides a list of the various components whose blood levels increase after administration of interferon orally. Of particular note, interferon-gamma blood levels were increased 7.8 fold. Interferon-gamma is known to elevate 26S proteasome levels. Thus oral administration of interferon-alpha will cause upregulation of 26S proteasome levels. In addition, as indicated in Table 1, the Ubiquitin-like 1 protein (sentrin) is increased by a factor of 2.6. Accordingly, the oral administration of low dose interferon-alpha results in the upregulation of components involved the degradation of cellular proteins, and enhancement of this system is anticipated to be efficacious in treating prion diseases.

Various changes in the details of the illustrated operational methods are possible without departing from the scope of the following claim s. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claim s, along with the full scope of equivalents to which such claim s are entitled. In the appended claim s, the terms “including” and “in which” are used as the plain-English equivalents of the respective terms “comprising” and “wherein.”

1. A method of treating bovine spongiform encephalopathy, said method comprising
identifying animals at risk of bovine spongiform encephalopathy
administering to said animals a composition comprising an effective amount of interferon-alpha.

2. The method of claim 1 wherein the interferon is administered orally.

3. The method of claim 2 wherein the interferon is admixed with the animals' water or feed.

4. The method of claim 1 wherein the amount of interferon-alpha is administered in a dosage of about 0.1 IU/lb to about 100 IU/lb of patient body weight.

5. The method of claim 4 wherein said composition further comprises trehalose.

6. The method of claim 5 wherein the composition further comprises a disaccharide selected from the group consisting of maltose, lactose and fructose.

7. The method of claim 6 wherein the disaccharide is anhydrous crystalline maltose.

8. A supplemented animal feed, comprising:
a feed supplement, wherein said feed supplement comprises
a type I interferon;
trehalose; and
a disaccharide selected from the group consisting of maltose, lactose and fructose;
and
a standard animal feed formulation, wherein said feed supplement is admixed with said standard animal feed formulation.

9. The supplemented animal feed of claim 8 wherein the type I interferon is interferon-alpha.

10. The supplemented animal feed of claim 9 wherein the disaccharide is anhydrous crystalline maltose.

11. The supplemented animal feed of claim 8 wherein the animal feed formulation is
for a ruminant species, said feed supplement being prepared in an encapsulated form, wherein
the encapsulated feed supplement bypasses the rumen before releasing its contents to the
digestive tract of the ruminant species.

12. A method of stablizing PrPc in a vertebrate species, said method comprising administering a composition to said vertebrate species, wherein the composition comprises interferon-alpha.

13. The method of claim 12 wherein the interferon is orally administered in a dosage amount of about 0.1 IU/lb to about 100 IU/lb of animal body weight.

14. The method of claim 12 wherein said composition further comprises
trehalose; and
a disaccharide selected from the group consisting of maltose, lactose and fructose.

15. The method of claim 14 wherein the disaccharide is anhydrous crystalline maltose.

16. The method of claim 12 wherein the composition is administered orally in a liquid form.

17. The method of claim 12 wherein the composition is administered in a dry form admixed with the animal's feed.

18. A method of inhibiting the transfer of BSE between cattle, said method comprising the step of administering a composition comprising interferon-alpha to the cattle.

19. The method of claim 18 wherein the interferon is administered in a dosage amount of about 0.1 IU/lb to about 100 IU/lb of animal body weight.

20. The method of claim 18 wherein said composition further comprises
trehalose; and
a disaccharide selected from the group consisting of maltose, lactose and fructose.

21. The method of claim 20 wherein the disaccharide is anhydrous crystalline maltose.

22. The method of claim 21 wherein the composition is administered in a dry form admixed with the cattles' feed.
